(19) United States
(12) Patent Application Publication
(10) Pub. No.: US 2025/0161656 A1
(43) Pub. Date: May 22, 2025

(54) VESSEL COMPLIANCE RESTORATION USING DUAL INFLATABLE SLEEVES

(71) Applicant: EDWARDS LIFESCIENCES CORPORATION, Irvine, CA (US)

(72) Inventor: Eran Goldberg, Nesher (IL)

(21) Appl. No.: 19/029,059

(22) Filed: Jan. 17, 2025

Related U.S. Application Data
(63) Continuation of application No. PCT/US2023/071224, filed on Jul. 28, 2023.
(60) Provisional application No. 63/370,901, filed on Aug. 9, 2022.

(51) Int. Cl. A61M 60/157; A61M 60/139; A61M 60/274; A61M 60/427; A61M 60/839; A61M 60/869

(52) U.S. Cl. CPC A61M 60/157; A61M 60/139; A61M 60/274; A61M 60/427; A61M 60/839; A61M 60/869

(57) ABSTRACT

A compliance restoration implant device includes a first compliant fluid container, a second compliant fluid container, and a conduit structure. The first compliant fluid container includes a first lumen that widens in response to fluid leaving the first compliant fluid container and narrows in response to the fluid entering the first compliant fluid container. The second compliant fluid container includes a second lumen that widens in response to the fluid leaving the second compliant fluid container and narrows in response to the fluid entering the second compliant fluid container. The conduit structure is coupled to the first compliant fluid container and the second compliant fluid container and is configured to pass fluid between the first compliant fluid container and the second compliant fluid container in response to changing pressure levels outside one of the fluid containers.

### RELATED APPLICATIONS

This application is a continuation of International Application No. PCT/US2023/071224, filed on Jul. 28, 2023, which claims the benefit of U.S. Provisional Application No. 63/370,901, filed on Aug. 9, 2022, the complete disclosures of which are hereby incorporated by reference in their entireties.

### BACKGROUND

The present disclosure generally relates to the field of medical implant devices. Insufficient or reduced compliance in certain blood vessels, including arteries such as the aorta, can result in reduced perfusion, cardiac output, and other health complications. Restoring compliance to such blood vessels can improve patient outcomes.

### SUMMARY

Described herein are devices, methods, and systems that facilitate the restoration of compliance characteristics to undesirably stiff blood vessels. Devices associated with the various examples of the present disclosure can include compliant body features in fluid communication with each other and configured to be positioned/disposed within an arterial blood vessel and a venous blood vessel to increase compliance of said vessels. In some examples, these compliant body features form dual inflatable sleeves that are filled with fluid that move from one sleeve to the other during systole and diastole.

For purposes of the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example representation of a heart and associated vasculature having various features relevant to one or more examples of the present inventive disclosure.

FIGS. 2A and 2B provide cross-sectional and side views, respectively, of a blood vessel experiencing compliant expansion during the systolic phase of the cardiac cycle.

FIGS. 3A and 3B provide cross-sectional and side views, respectively, of the artery shown in FIGS. 2A and 2B during the diastolic phase of the cardiac cycle.

FIG. 4 is a graph illustrating blood pressure over time in an example healthy patient.

FIG. 5 is a graph illustrating blood pressure over time in an example patient having reduced aortic compliance.

FIG. 6 is a horizontal cross-sectional view of a compliance restoration device implanted in arterial and venous blood vessels in accordance with one or more examples.

FIGS. 7A and 7B illustrate horizontal cross-sectional views of the compliance restoration device during diastole and systole, according to one or more examples.

FIGS. 8A and 8B illustrate cross-sectional views across a blood vessel containing a compliant fluid container during expansion and contraction, according to one or more examples.

FIG. 9 illustrates a perspective view of the compliance restoration device, according to one or more examples.

FIG. 10 provides a top-down view of the conduit and examples of port structures that can be used to anchor the compliance restoration device to the blood vessels, according to one or more examples.

FIG. 11 is a flow diagram illustrating a process for implanting a compliance restoration device in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Compliance and Anatomy

Certain examples are disclosed herein in the context of vascular implant devices, and in particular, compliance restoration implant devices implanted in the aorta and/or inferior vena cava. However, although certain principles disclosed herein may be particularly applicable to the anatomy of the aorta and inferior vena cava, it should be understood that compliance restoration implant devices in accordance with the present disclosure may be implanted in, or configured for implantation in, any suitable or desirable blood vessels or other anatomy.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart, which is discussed in detail below.

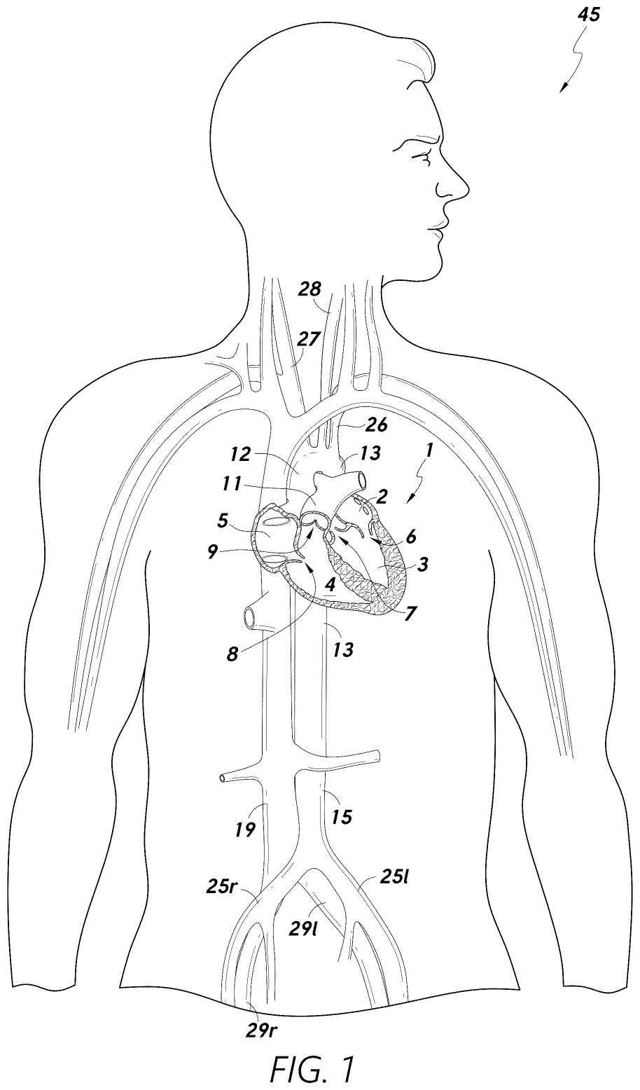

FIG. 1 illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11.

The pulmonary artery 11 carries deoxygenated blood from the right side of the heart to the lungs. The pulmonary artery 11 includes a pulmonary trunk and left and right pulmonary arteries that branch off of the pulmonary trunk, as shown. In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (i.e., mitral and tricuspid) heart valves generally are coupled to a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17, referred to as the septum, separates the left 2 and right 5 atria and the left 3 and right 4 ventricles.

The vasculature of the human body, which may be referred to as the circulatory system, cardiovascular system, or vascular system, contains a complex network of blood vessels with various structures and functions and includes various veins (venous system) and arteries (arterial system). Both arteries and veins are types of blood vessels in the cardiovascular system. Generally, arteries, such as the aorta, carry blood away from the heart, whereas veins, such as the inferior and superior venae cavae, carry blood back to the heart.

As mentioned above, the aorta is coupled to the heart 1 via the aortic valve 7, which leads into the ascending aorta 12 and gives rise to the innominate artery 27, the left common carotid artery 28, and the left subclavian artery 26 along the aortic arch before continuing as the descending thoracic aorta 13 and further the abdominal aorta 15. References herein to the aorta may be understood to refer to the ascending aorta (also referred to as the “ascending thoracic aorta”), aortic arch, descending aorta, thoracic aorta (also referred to as the “descending thoracic aorta”), abdominal aorta, or other arterial blood vessel or portion thereof.

Arteries, such as the abdominal aorta 15, may utilize blood vessel compliance (e.g., vessel compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner that affects blood flow in response to increasing transmural pressure, or the tendency of a blood vessel (e.g., artery) or prosthetic implant device, or portion thereof, to resist recoil toward its original dimensions on application of a distending or compressing force. Compliance of a blood vessel or prosthetic implant device may or may not be based on elasticity or stretchability of the blood vessel walls. Compliance of a prosthetic implant device may or may not be based on changing a size of a lumen to regulate blood flow.

Vessel compliance facilitates perfusion of organs in the body with oxygenated blood from the heart, Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient vessel compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on vessel compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

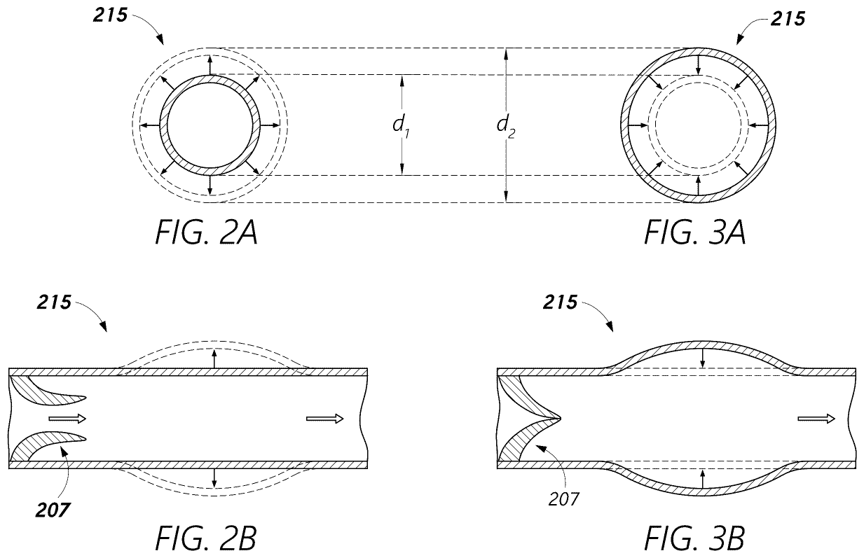

FIGS. 2A and 2B provide cross-sectional and side views, respectively, of a blood vessel 215, such as an artery (e.g., aorta), experiencing expansion during the systolic phase of the cardiac cycle. As understood by those having ordinary skill in the art, the systolic phase of the cardiac cycle is associated with the pumping phase of the left ventricle, while the diastolic phase of the cardiac cycle is associated with the resting or filling phase of the left ventricle. As shown in FIGS. 2A and 2B, with proper vessel compliance, an increase in volume will generally occur in an artery when the pressure in the artery is increased. With respect to the aorta, as shown in FIGS. 2A and 2B, as blood is pumped into the aorta 215 through the aortic valve 207, the pressure in the aorta increases and the diameter of at least a portion of the aorta expands. A first portion of the blood entering the aorta 215 during systole may pass through the aorta during the systolic phase, while a second portion (e.g., approximately half of the total blood volume) may be stored in the expanded volume caused by vessel compliance, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of vessel compliance may have a significant effect on perfusion and/or blood pressure in some patients. For example, arteries with relatively higher compliance may be conditioned to more easily deform than lower-compliance arteries under the same pressure and/or volume conditions. Compliance (C) may be calculated using the following equation, where ΔV is the change in volume (e.g., in mL), and ΔP is the pulse pressure from systole to diastole (e.g., in mmHg):

$$C = \frac{Δ V}{Δ P} \qquad ( 1 )$$

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

Vessel compliance restoration devices, methods, and concepts disclosed herein may be generally described in the context of the thoracic and/or abdominal aorta. However, it should be understood that such devices, methods and/or concepts may be applicable in connection with any other artery or blood vessel.

FIGS. 3A and 3B provide cross-sectional and side views, respectively, of the artery 215 shown in FIGS. 2A and 2B during the diastolic phase of the cardiac cycle. As shown, vessel compliance may cause retraction of the blood vessel wall inward during diastole, thereby creating pressure to continue to push blood through the artery 215 when the valve 207 is closed. For example, during systole, approximately 50% of the blood that enters the artery 215 through the valve 207 may be passed through the artery, whereas the remaining 50% may be stored in the artery, as enabled by expansion of the vessel wall. Some or all of the stored portion of blood in the artery 215 may be pushed through the artery by the contracting vessel wall during diastole. For patients experiencing arterial stiffness (i.e., lack of compliance), their arteries may not operate effectively in accordance with the expansion/contraction functionality shown in FIGS. 2A and 2B and FIGS. 3A and 3B.

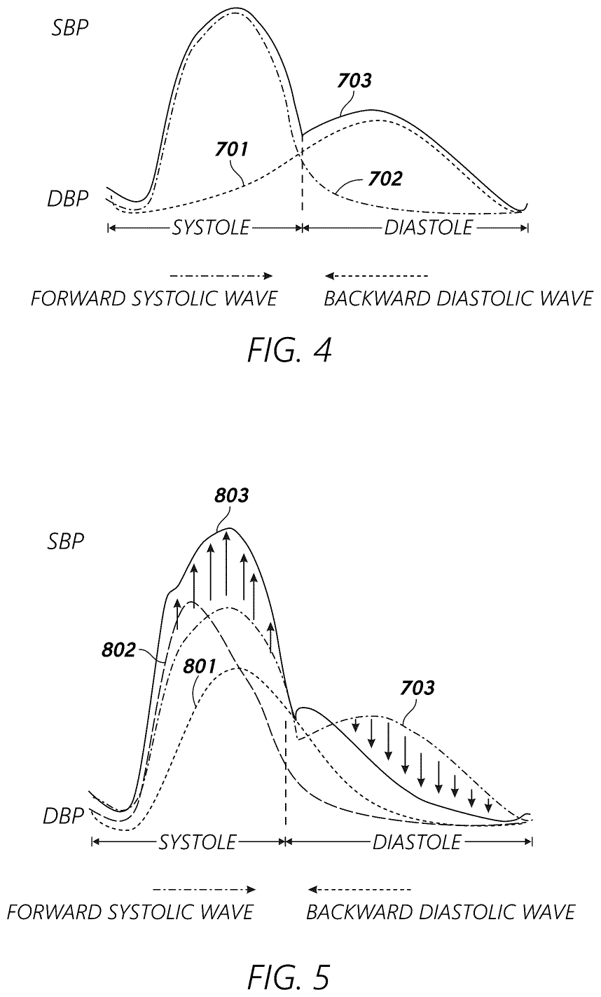

FIG. 4 is a graph illustrating blood pressure over time in an example healthy patient, wherein arterial blood pressure is represented as a combination of a forward systolic pressure wave 701 and a backward diastolic pressure wave 702. The combination of the systolic wave 701 and the diastolic wave 702 are represented by the waveform 703.

FIG. 5 is a graph illustrating blood pressure over time in an example patient having reduced aortic compliance. The graph of FIG. 5 shows, for reference purposes, the example combined wave 703 shown in FIG. 4. When low compliance is exhibited, less energy may be stored in the aorta compared to a healthy patient. Therefore, the systolic waveform 802 may demonstrate increased pressure relative to a patient having normal compliance, while the diastolic waveform 801 may demonstrate reduced pressure relative to a patient having normal compliance. Therefore, the resulting combined waveform 803 may represent an increase in the systolic peak and a drop in the diastolic pressure, which may cause various health complications. For example, the change in waveform may impact the workload on the left ventricle and may adversely affect coronary profusion.

In view of the health complications that may be associated with reduced vessel compliance, as described above, it may be desirable in certain patients and/or under certain conditions, to at least partially alter compliance properties of the aorta or other artery or blood vessel in order to improve cardiac and/or other organ health. Disclosed herein are various devices and methods for at least partially restoring compliance to a blood vessel, such as the aorta. Certain examples disclosed herein achieve the effect of vessel compliance through the use of implantable compliant fluid containers, which can be used to regulate blood flow to mimic the effects of compliant vessels. For example, a compliance restoration device in accordance with the present disclosure may comprise a pair of compliant fluid containers that are fluidly connected and can be placed in blood vessels, such as an arterial vessel and an adjacent vein. The device can include a conduit structure that is configured to support/maintain port openings through the artery and vein walls to provide fluid communication between the compliant fluid container in the artery and the compliant fluid container disposed within the vein. The device may be anchored to the blood vessel wall using any suitable type of anchor means, such as a wire-form or stent anchor. Although certain examples of compliance restoration devices are described herein in the context of deployment in the aorta and in one of the inferior or superior vena cava, it should be understood that compliance restoration devices in accordance with the present disclosure may be deployed in any major artery or vein that may benefit from increased compliance characteristics,

### Compliance-Restoration Implant Devices

The present disclosure relates to systems, devices, and methods for restoring the effect of compliance to the aorta and/or other arterial and/or venous blood vessel(s) to provide improved perfusion of the heart muscle and/or other organ(s) of the body. For example, examples of the present disclosure can include compliant tubular or sleeve-like devices configured to modulate blood flow in the aorta and/or other arterial blood vessel and in the inferior/superior vena cava and/or other venous blood vessel.

By modulating blood flow through a pair of compliant fluid containers disposed at least in part within a venous and arterial blood vessel, examples of the present disclosure can increase vessel compliance in a manner that presents a reduced risk of clotting/embolism formation compared to certain other compliance restoration solutions. Furthermore, with the compliant fluid containers disposed within blood vessels, as opposed to external to the blood vessel(s), incidences of leakage and/or rupture of the container may be contained within the blood vessel(s), thereby reducing hazards associated with extravascular blood leakage, such as within the abdominal and/or chest cavity. Rather, such leakage may be deposited within the vascular system, resulting in little or no harm to the patient. Furthermore, devices disclosed herein can be implanted using a transcaval delivery/access, thereby allowing for delivery system components and/or other working instruments to be advanced through the venous system (e.g., inferior vena cava), rather than the arterial system, which can allow for relatively larger-profile devices/systems to be used and/or otherwise provide a relatively safer access and procedural implementation for implantation of the device(s).

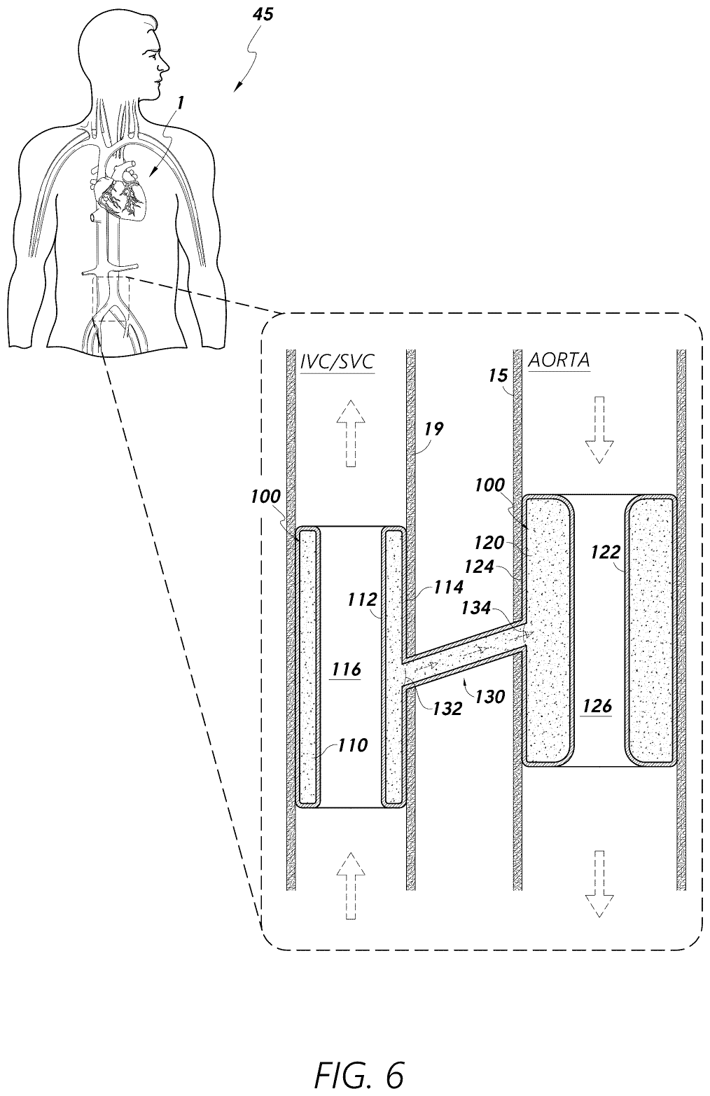

FIG. 6 is a horizontal cross-sectional view of a compliance restoration device 100 implanted in arterial 15 (e.g., aorta) and venous 19 blood vessels (e.g., inferior or superior vena cava “IVC/SVC”) in accordance with one or more examples. The compliance restoration implant device 100 is shown as implanted in a manner as to modulate blood in the arterial 15 and venous blood vessels 19 in a location around the abdomen, though other locations can also be used. The example compliance restoration implant device 100 includes a venous compliant fluid container 110, an arterial compliant fluid container 120, and a conduit 130 joining the venous and arterial compliant fluid containers.

For case of reference, the disclosure may distinguish between the venous or arterial compliant fluid containers, but that does necessarily mean that the two fluid containers differ from each other. In some examples, the compliant fluid containers are identical and “venous” and “arterial” merely refer to their implanted location. Likewise, the disclosure may refer to a first or second compliant fluid container or other structure of the compliance restoration device 100, but the ordinal numbering does not denote a particular order of implantation or specific location.

In one example, the compliant fluid containers are fillable, tubular structures forming lumens that allow blood to flow through. The venous compliant fluid container 110 can include a first inner wall 112 and a first outer wall 114. The first inner wall 112 can circumferentially form a first lumen 116 through which blood passes. As the venous compliant fluid container 110 fills up, the first lumen 116 narrows, restricting the blood flowing through. As fluid leaves the venous compliant fluid container 110, the first lumen 116 widens, allowing blood to more easily flow through. Likewise, the arterial compliant fluid container 120 can include a second inner wall 122 and a second outer wall 124. The second inner wall 122 can circumferentially form a second lumen 126 through which blood passes. As the arterial compliant fluid container 120 fills up, the second lumen 126 narrows, restricting the blood flowing through. As fluid leaves the arterial compliant fluid container 120, the second lumen 126 widens, allowing blood to more easily flow through.

The compliance restoration device 100 may include a first port structure 132 and a second port structure 134 configured to provide fluid access to the venous and arterial compliant fluid containers 110, 120 respectively. In one example, the conduit 130 is connected to the first port structure 132 on one end and to the second port structure 134 on its other end, with the conduit 130 passing through a first opening in the venous blood vessel 19 wall and a second opening in the arterial blood vessel. Fluid can then move from the venous compliant fluid container 110, through the first port structure 132, through the conduit 130, through the second port structure 134 and to the arterial compliant fluid container 120.

The port structures 132, 134 may be distinct from the compliant fluid containers or integrated. For example, the port structures may be stent-like devices for anchoring the conduit to a compliant fluid container. In one scenario, the port structures are distinct structures that are then added to the compliant fluid container when attaching the conduit. In another example, the port structures are built-in to the compliant fluid containers, such as valves and attachment points (e.g., threaded connector, friction fit tube, etc.). In another example, the venous and arterial compliant fluid containers 110, 120, first port structure 132, 134 (e.g., an opening in the outer wall of a fluid container), and the conduit 130 are one integral piece that are already connected together when implanting the compliance restoration device 100 into the blood vessels. In another example, the venous and arterial compliant fluid containers 110, 120 are already conjoined via the conduit 130 and the port structures are the intersection formed by conduit at each end when joining with one of the fluid containers. These T-intersections can serve to anchor the venous and arterial compliant fluid containers 110, 120 to the openings through the blood vessels through which the conduit passes through.

One of the compliant fluid containers can reside inside the venous blood vessel 19 (e.g., inferior vena cava), where pressure may be generally lower compared to the arterial blood vessel 15 (e.g., aorta), while the other can reside in the arterial blood vessel 15. In this particular example, the venous compliant fluid container 110 is implanted in the inferior vena cava (IVC) or superior vena cava (SVC). For ease of reference, the disclosure sometimes refers to the aorta 15 and the vena cava vessel 19 as the locations in which the fluid containers are positioned, where the vena cava vessel can refer to either the IVC or SVC. However, other venous blood vessels could also be used.

Generally, a pressure gradient may exist between the arterial blood vessel 15 and the venous blood vessel 19, wherein the pressure level within the arterial blood vessel 15 is greater than the pressure within the venous blood vessel 19 through at least a portion of the cardiac cycle. Due to the differences in pressure between the locations of each of the compliant fluid container, one fluid container will expand while the other contracts, generally until some equilibrium is reached. One compliant fluid container may advantageously be configured to expand with respect to one or more dimensions and store energy that is released when the other fluid container contracts or otherwise deforms in response to changes in pressure in the arterial 15 and/or venous 19 blood vessels during systole/diastole. These compliant fluid containers can then alternate expansion/contraction as the pressure differential grows or lessens.

The arterial blood pressure for a healthy person is typically around 120 mm HG during systole and around 80 mm HG during diastole, but can get higher (e.g., 160 mmHg during systole) for a patient suffering from reduced aortic compliance (“aortic stiffness”). In one example scenario, during systole, the high-pressure forces presses on the container 120 wall and pushes fluid out of the arterial compliant fluid container 120 and into the venous compliant fluid container 110. This causes the venous compliant fluid container 110 to expand while the arterial compliant fluid container 120 contracts, allowing the arterial blood vessel 15 to accommodate more blood volume, generating aortic compliance. As the pressure lessens during diastole, the compliant and/or elastic nature of the of the venous compliant fluid container 110 can cause it to revert back to a smaller size, pushing fluid back into the arterial compliant fluid container 120 and reducing the blood vessel 15 blood volume. As the pressure in the arterial blood vessel 15 is less than during diastole, there is less resistance against the arterial compliant fluid container 120, allowing it to expand and accept the fluid from the venous compliant fluid container 110.

In one scenario, in response to diastole in the aorta 15, the arterial fluid container 120 expands by receiving fluid via the conduit 130. Meanwhile, during diastole in the vena cava vessel 19, the venous fluid container 110 compresses by sending the fluid to the arterial fluid container via the conduit 130. As a result, the lumen 116 in the venous fluid container 110 narrows. In response to systole in the aorta 15, the arterial fluid container 120 compresses by sending the fluid via the conduit to the venous fluid container. Meanwhile, during systole in the vena cava vessel 19, the venous fluid container 110 expands by receiving fluid from the arterial fluid container 120 via the conduit. This expansion can serve as a form of energy storage, as the expanded container can have a natural elastic tendency to compress back to its original size. This elastic force can be used later on to push fluid back to the arterial fluid container 120 (e.g., during diastole).

In some examples, anchoring devices (not shown) may be deployed to maintain the positions of the compliant fluid containers 110, 120 in their respective locations in the arterial wall 79 and the arterial blood vessel 15 and the venous blood vessel 19. Different types of wall anchor structure(s) can be used, such as memory metal frames, suturing, or the like. The conduit 130 may also act as an anchor by keeping the compliant fluid containers located around the hole in the vessel walls. As the conduit 130 extends through the vessel wall via the hole, the conduit 130 can prevent or limit movement of the compliant fluid containers relative to the hole in the vessel wall.

The blood flows from the heart, to arterial blood vessel 15, through the lumen 126 of the arterial compliant fluid container 120, and returning back to the vena cava vessel 19, through the lumen 116 of the venous compliant fluid container 110, as indicated by the illustrated arrows in FIG. 6. By placing the venous compliant fluid container 110 in the venous blood vessel 19 and the arterial compliant fluid container 120 in the arterial blood vessel 15, compliance or the effect of compliance can be added back to those blood vessels. For example, as pressure increases within the arterial blood vessel 15, blood flow enters the lumen 126 of the arterial compliant fluid container 120, thereby causing the compliant bypass structure to deform or otherwise shrink due to the compliant and/or elastic characteristics thereof. The lumen 126 can then enlarge, allowing blood to flow through at a lower pressure. As pressure in the arterial blood vessel 15 decreases, energy stored in the venous compliant fluid container 110 due to expansion thereof can cause the venous compliant fluid container 110 to contract, thereby pushing fluid out of the venous compliant fluid container 110 and back into the arterial compliant fluid container 120. As a result of the contraction, the lumen 116 in the venous compliant fluid container 110 can enlarge, allowing blood to flow through at a lower pressure and/or providing greater volume for blood to pool in the venous vessel. The changes in the lumens 116, 126 can recreate the effect of compliance (e.g., modulation of blood flow) in the arterial system. Furthermore, expansion of the venous compliant fluid container 110 within the venous blood vessel 19 can increase pressure in the venous blood vessel 19 and/or push fluid disposed therein in a manner as to increase compliance and/or flow within the venous system to some degree. Therefore, the compliance restoration device 100 can serve to increase compliance in both the arterial and venous systems of the circulatory system of the patient.

By implanting the device 100 such that the venous and arterial compliant fluid containers 110, 120 are disposed withing the blood vessels, in the event that the compliance restoration device 100 leaks or ruptures in some manner, such leakage may be maintained substantially within the circulatory system. Such leakage within the blood vessel(s), particularly if the fluid within the fluid containers is saline or similarly absorbable fluid, may result in relatively less damage/injury to the patient compared to leakage outside of the circulatory system within the body cavity.

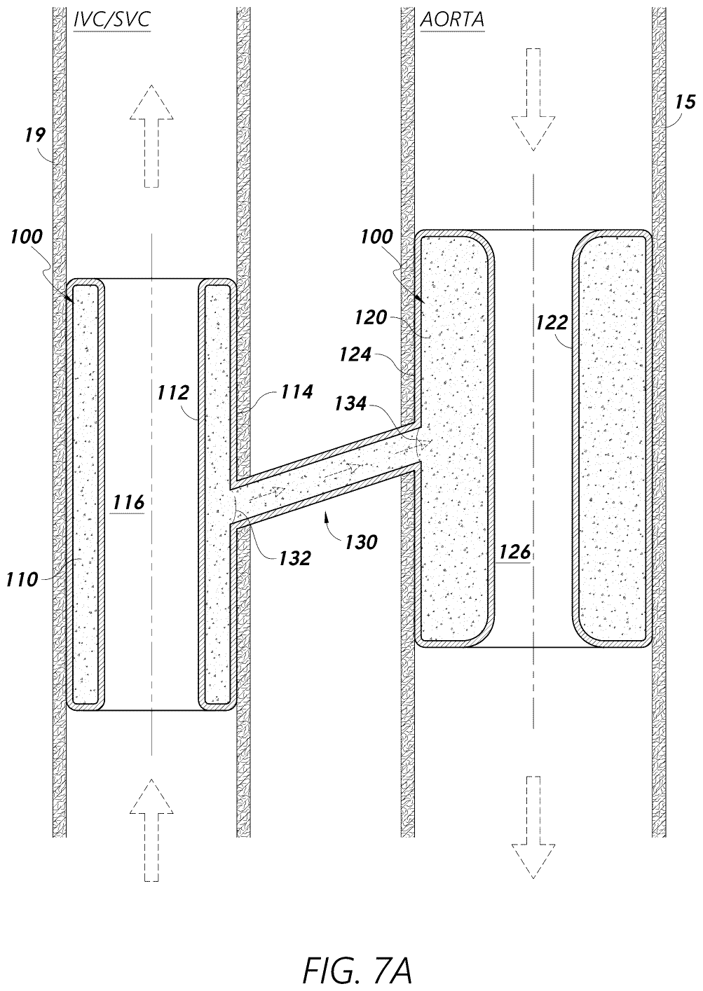

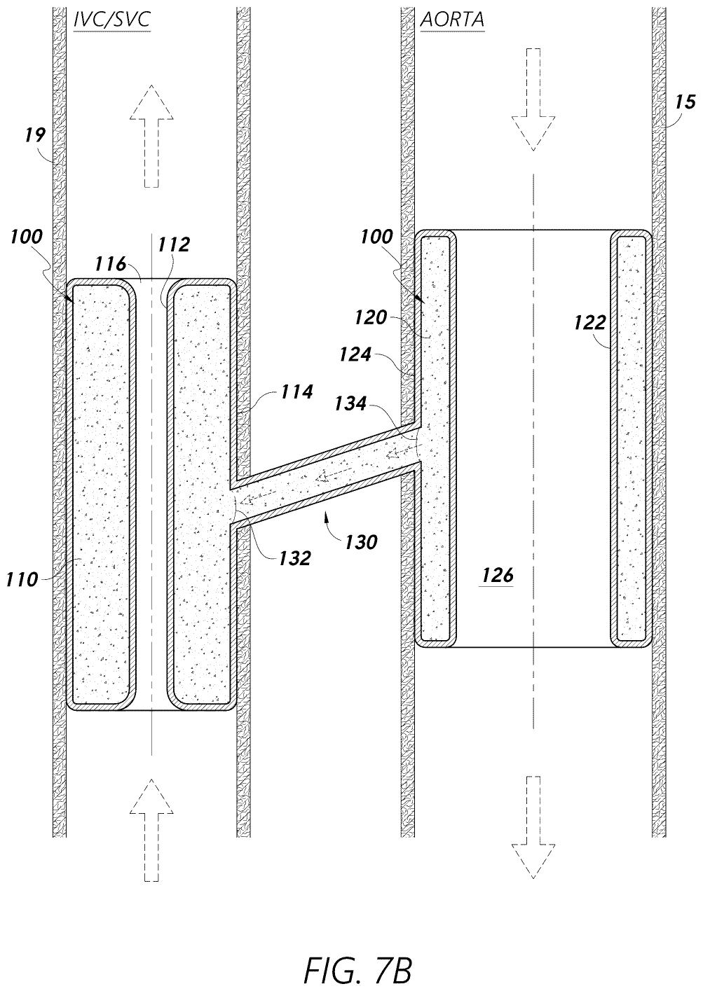

FIGS. 7A and 7B illustrate a cross-sectional view of the compliance restoration device 100 during diastole and systole, according to certain examples. The venous compliant fluid container 110 includes a first lumen 116 through which blood flows towards the heart, as indicated by the arrows in FIGS. 7A and 7B. The arterial compliant fluid container 120 includes a second lumen 126 through which the blood flows away from the heart, as indicated by the arrows. In FIG. 7A, the aortic pressure is low during diastole, reducing or removing pressure coming from the arterial complaint fluid container 120 and allowing the venous fluid container 110 to contract. The contraction of the venous fluid container 110 causes the fluid within the compliance restoration device 100 to travel from the venous compliant fluid container 110, through the conduit 130, into the arterial compliant fluid containers 120. As a result, the first lumen 116 widens while the second lumen 126 narrows, providing greater space for blood to pool in the vena cava vessel 19 and/or pushing blood in the aorta 15 as arterial compliant fluid containers 120 squeezes the blood within the second lumen 126.

While the FIG. 7A shows that the first lumen 116 is larger than the second lumen 126 when the venous compliant fluid container 110 is in its contracted state, that is not necessarily the case depending on the configuration of the compliance restoration device 100. For example, the one fluid container may be sized to be smaller, larger, or equal to the other during its contracted state, depending on the desired behavior. Different relative sizes between the fluid containers may provide additional control on the level of blood flow in the venous and arterial vessels.

The compliance restoration device 100, in particular the compliant fluid containers 110, 120, can be constructed of a compliant material, such as an elastomeric polymer, nylon, rubber, or other material. In some examples, the compliance restoration device 100 comprises biological tissue in addition to, or as an alternative to, a polymer or elastomeric material. For example, bovine pericardial tissue may be utilized to form the fluid containers, wherein a secondary structure, such as a memory metal braid or frame, may be secured around the compliant fluid containers 110, 120 to allow the compliance restoration device 100 to expand/stretch and retract/recover, as necessary to reintroduce compliance to the circulatory system. Other implementations may use different materials with different elastic characteristics. For example, a spring mechanism may be wrapped around one or more containers to provide elasticity, while the containers themselves may be non-elastic or only somewhat elastic, broadening the possible materials that can be used for the containers.

The elastic/compliant characteristics of the compliant fluid containers 110, 120 may advantageously increase compliance in a manner as may not be achievable without such elastic/compliant characteristics. For example, in the absence of elastic/compliant features, the compliant fluid containers 110, 120, without the ability to change in volume in response to increases in pressure therein, may simply serve to narrow the blood vessel passageway without absorbing and returning energy to/from the system and/or resulting in a change in volume of the vasculature throughout the cardiac cycle, which may generally not improve compliance. In some examples, the compliant fluid containers may use the same materials. In other examples, one compliant fluid container may use more or different materials to increase the elastic/compliant characteristic relative to the other compliant fluid container. For example, as pressure is generally higher in the arterial vessel, the venous compliant fluid container 110 may need greater contractive strength to push the fluid into the arterial compliant fluid container 120 against the greater pressure. Some implementations may use a spring or memory metal frame around the container 120 to provide greater elasticity.

The compliance restoration device 100 can be substantially fluid-tight, such that blood cannot permeate the walls of the compliance restoration device 100. For example, such fluid tightness may facilitate the elastic expansion or other deformation of the structure in the presence of increased fluid pressure therein, which serves to increase the compliance-restoring characteristics of the compliance restoration device 100. FIG. 7B illustrates an example of the compliance restoration device 100 during systole, when pressure is generally highest in the aorta 15. The higher pressure causes the fluid within the compliance restoration device 100 to travel from the arterial compliant fluid container 120, through the conduit 130, into the venous compliant fluid containers 110, causing it to expand. As a result, the second lumen 126 widens while the first lumen 116 narrows, allowing greater blood flow in the aorta 15.

By varying the size of the passageway for the blood through the compliance restoration device 100, the compliance restoration device 100 can mimic the behavior of healthy blood vessels. For example, healthy arterial vessels have a tendency to stretch in response to higher pressure, such as during systole. The greater the compliance of an artery, the more effectively it is able to expand to accommodate surges in blood flow without increased resistance or blood pressure. However, less compliant blood vessels will not stretch much, leading to higher blood pressure. The compliance restoration device 100 can substitute for this vessel stretchability by changing the lumen size to account for greater blood flow during systole, while maintaining blood pressure or at least limiting its rise. As pressure increases in the blood vessel, the pressure differential pushes the fluid in the compliance restoration device 100 into the compliant fluid container with lower surrounding pressure. In the higher-pressure blood level, the lumen size increases as the compliant fluid container shrinks, accommodating the greater blood flow while limiting the rise in blood pressure in that vessel.

Although the venous and arterial compliant fluid containers 110, 120 are illustrated as tubular structures, it should be understood that these containers may have any suitable or desirable shape or form. For example, the compliant fluid containers 110, 120 may have a pouch-type form that may not necessarily be tubular in shape. In that example, the fluid containers can partially occlude the blood vessels and can change the blood flow in response to pressure differentials by either growing to block more of the vessel passageway or shrinking to expand the vessel passageway.

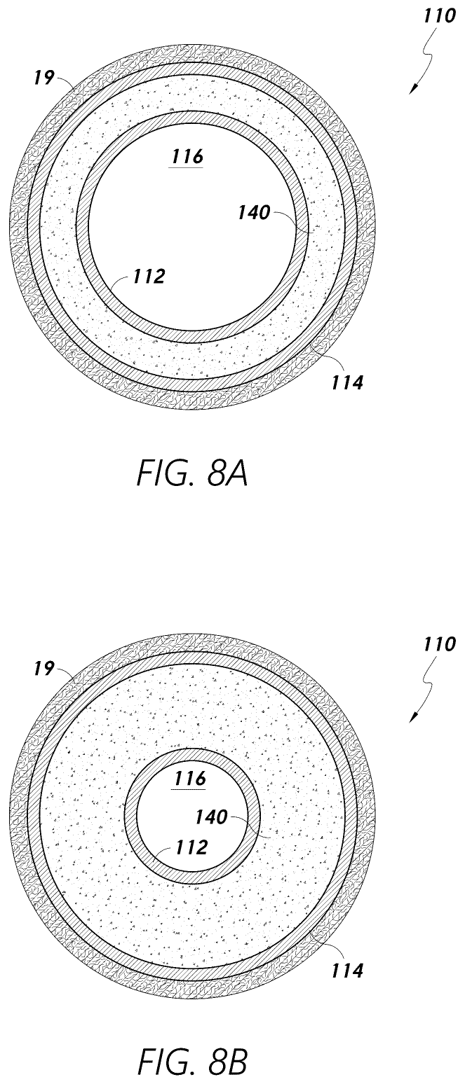

FIGS. 8A and 8B illustrate cross-sectional views across a blood vessel containing a compliant fluid container during expansion and contraction, according to certain examples. For ease of explanation, the figures refer to the vena cava vessel 19 and the venous compliant fluid container 110, though the following can similarly apply to the arterial vessel 15 and the arterial compliant fluid container 120. In FIG. 8A, the outer wall 114 of venous compliant fluid container 110 abuts against the inner surface of the vena cava vessel 19. The outer wall 114 may be pressed against the surface of the vessel or may leave one or more gaps between the wall and the vessel surface. The inner wall 112 of the venous compliant fluid container 110 forms the first lumen 116. In between the inner wall 112 and the outer wall 114 is a space or reservoir for fluid 140. In the illustrated example, lower pressure in the arterial vessel 19 and/or the contraction of the venous compliant fluid container 110 has pushed most of the fluid 140 out of the venous compliant fluid container 110 and into the other compliant fluid container. As a result, the first lumen 116 is in an enlarged state, providing a wider passageway for blood flow within the vessel and/or providing a larger space for blood to be stored in the vena cava vessel 19.

FIG. 8B shows the first lumen 116 in a shrunken or narrowed state, providing a narrower passageway for blood flow within the vessel. In the illustrated example, the pressure differential (e.g., during systole) has pushed most of the fluid 140 into the venous compliant fluid container 110 from the other compliant fluid container, causing the venous compliant fluid container 110 to enlarge and to narrow the first lumen 116. This can cause the venous compliant fluid container 110 to squeeze the blood within the first lumen 116 and cause at least some additional blood flow.

The venous and arterial compliant fluid containers 110, 120 may be sized and/or configured, such as with respect to a cross-sectional diameter thereof in one or more portions of the structure, such that the fluid containers 110, 120 do not occlude the blood vessels in a disadvantageous manner. Alternatively, the compliant fluid containers 110, 120 may be sized and/or dimensioned such that they substantially occludes the blood vessels in which they are implanted in one or more periods of the cardiac cycle. In some examples, the compliant fluid containers 110, 120 are constructed in a manner as to limit expansion thereof in response to increasing pressure conditions, such that the compliant fluid containers 110, 120 do not expand to a degree that causes undesired occlusion of the blood vessels. For example, the expandability of the fluid containers 110, 120 may have a structural limit beyond which it will not expand further regardless of increases of pressure therein.

During implantation, an opening can be made through the vessel wall. The compliance restoration device 100 (or a portion of it, such as one of the compliant fluid containers) may pass through the opening through the walls of the artery 15 and adjacent vein (e.g., inferior vena cava) 19, respectively. Implantation may also be done in the opposite order, starting from the adjacent vein 19. The term “opening” is used herein according to its broad and ordinary meaning. With respect to implant devices of the present disclosure as implanted in one or more blood vessels, the term “opening” may refer to an opening within an aortic blood vessel, a venous blood vessel, and/or the combination of an opening through both an arterial blood vessel wall and an at least partially over lapping opening in a venous blood vessel wall, such that the overlap of the openings provides a single opening through both blood vessel walls. The opening may be maintained by the port structure, which may have any suitable or desirable structure or form, such as a stent, shunt and/or other structure.

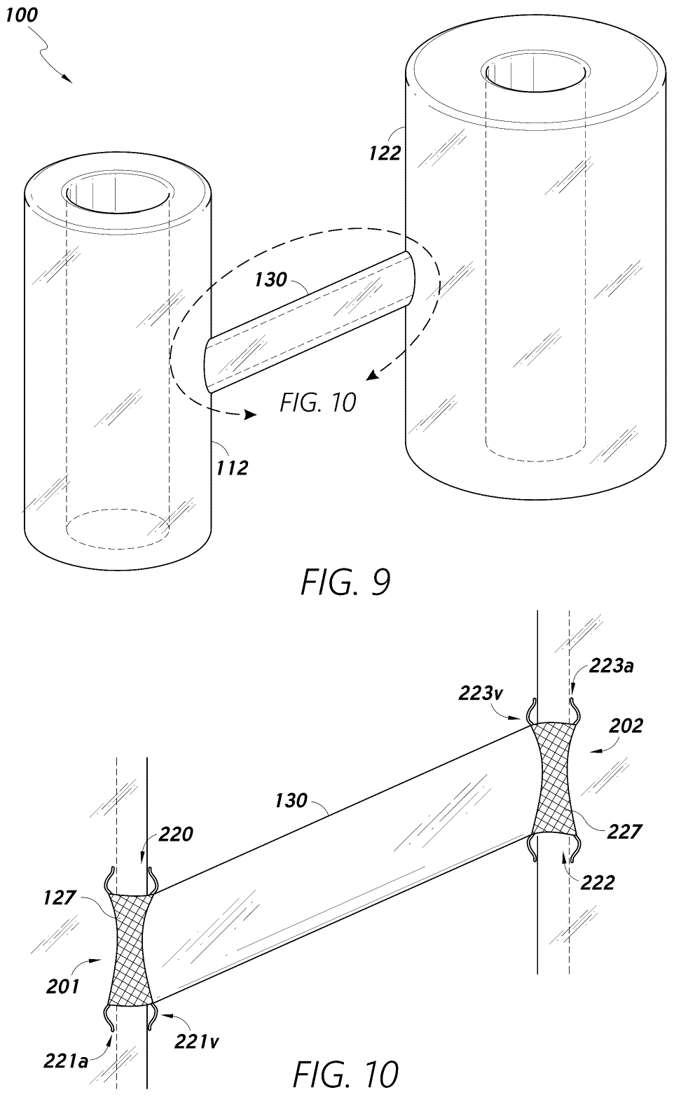

FIG. 9 illustrates a perspective view of the compliance restoration device 100, according to one or more examples. In the illustrated example, the compliance restoration device 100 includes a first compliant fluid container 110, a second compliant fluid container 120, and a conduit 130. Prior to implantation, the first compliant fluid container 110 and the second compliant fluid container 120 may already be joined together by the conduit. In other scenarios, the first and second compliant fluid container 110, 120 can be joined by the conduit 130 after implantation in the body.

FIG. 10 provides a top-down view of the conduit 130 and examples of port structures 201, 202 that can be used to anchor the compliance restoration device 100 to the blood vessels, according to one or more examples. The device 100 can be anchored in the wall of the arterial blood vessel 15 and/or the wall of the venous blood vessel 19 (e.g., IVC or SVC) using any suitable or desirable anchoring means, such as one or more contact arms, flanges, grommets, sutures, tabs, hoops, wire forms, barbs, and/or the like. For example, the port structures 201, 202 may be a woven structure, such as a woven memory metal braided structure, or the like. Although illustrated with one or more port structures 201, 202 using anchoring structures, it should be understood that implant devices of the present disclosure may be implanted without including anchoring structures.

In the illustrated example, the port structures 201, 202 ports can be reinforced with respective stent frames 227, which may form at least part of respective port/anchor structures 220, 222. For example, the stent frames 227 can comprise self-expanding memory metal frames that are configured to expand to form a suitable fluid seal within blood vessel wall openings, as described herein. Furthermore, the frames 227 can serve to approximate the arterial 79 and venous 78 walls when implanted. For example, when implanted, the anchor structures 221, 222, 223 can hold the vessel walls together in some manner to cause such blood vessel walls to be approximated to one another, thereby reducing the risk of fluid leakage outside of the vasculature.

Flow control for compliance restoration implant devices in accordance with aspects of the present disclosure may be achieved through the use of port structures having certain absolute and/or relative sizes. For example, a first port structure 201 may be configured with a flow channel area having a diameter or other dimension D1 that is greater than a corresponding diameter/dimension D2 associated with a second port structure 202. For example, in some examples, a first port structure may have a diameter of approximately 2-3 cm, whereas a second port structure may have a diameter of approximately 1-2 cm or vice versa. With a relatively enlarged first port structure, the pressure associated with blood flow through the first port structure 201 may be relatively lower compared to the blood flow through the second port structure 202 support structure, thereby promoting stronger blood flow in one direction than the other.

### Compliance-Restoration Device Implantation Processes

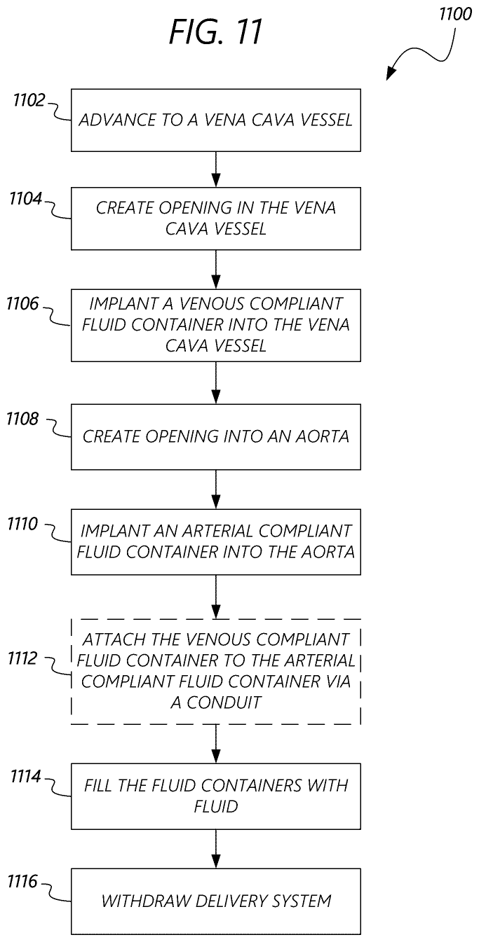

FIG. 11 is a flow diagram illustrating a process 1100 for implanting a compliance restoration device 100 in accordance with one or more examples. For ease of explanation, the following refers to components described in earlier figures. While the following scenario describes one possible order, other scenarios may have events occurring in a different order. For example, the arterial compliant fluid container 120 may be disposed in the aorta 15 before the venous compliant fluid container is disposed in the vena cava vessel 19.

At block 1102, the process 1100 involves advancing one or more delivery system components containing the compliance restoration device 100 to a location at a vena cava vessel 19 (e.g., IVC). Such an approach can be made via one of the iliac veins 29. For example, the compliance restoration device may be contained within a delivery catheter in a crimped or otherwise compressed configuration to allow for transportation thereof transvascularly. For example, a guidewire may be introduced into the femoral vein and further into the inferior vena cava through a percutaneous access. Other approaches may also be used to advance to the location in the vena cava vessel 19.

At block 1104, the process 1100 involves creating an opening in the vena cava vessel, for example, by puncturing the walls of the vena cava vessel 19 (or other venous blood vessel if deployed there). If the approach is made from within the vena cava vessel 19, the puncture is made from the inside out. However, in some situations, the approach may be made externally from the vena cava vessel 19 and the puncture is made from the outside in.

At block 1106, the process 1100 involves implanting a venous compliant fluid container 110 into the vena cava vessel 19. Deploying the venous compliant fluid container 110 may involve anchoring the container in the vessel. Such anchor features or means may be a first port structure of the compliance restoration device 100 being attached against and/or to the wall of the vena cava vessel 19.

For example, once the delivery system has crossed over into the vena cava vessel 19, anchor feature(s) may be deployed from the catheter/sheath, wherein such anchor feature(s) may serve to retain the implant device in a manner as to resist the device being pulled back through the opening in the blood vessel walls. For example, the anchor features may comprise one or more hooks, barbs, flanges, arms, clamps, tabs, sutures, and/or the like.

At block 1108, the process 1100 involves creating an opening in an aorta 15, for example, by puncturing the walls of the vessel 15 (or other arterial blood vessel if deployed there). In one example, the approach is after exiting the vena cava vessel 19. However, other approaches are possible. If the approach is made from within the aorta 15, the puncture is made from the inside out. If the approach is made externally from the aorta 15, the puncture is made from the outside in.

In some scenarios, the puncture may be made concurrently through the vena cava vessel 19 and the aorta 15 to advance one or more of the delivery system components into the aorta 15. For example, for implantation of the compliance restoration device in accordance with aspects of the present disclosure in the abdominal space of a patient, a transcaval procedure may be implemented, wherein access to the aorta is made via the inferior vena cava by puncturing the blood vessel walls separating the arterial and venous blood vessels and advancing the delivery system through the opening formed therein. Transcaval procedures may be preferable when implanting devices disclosed herein for patients presenting anatomical conditions in which the arterial system is difficult to access and/or navigate within. For example, relatively small, tortuous, and/or heavily calcified aortas can be better suited for transcaval access. Furthermore, pressure conditions in the arterial system may make it difficult or untenable to access the aorta via the femoral artery or other arterial access. In some implementations, fluoroscopy or other imaging technology may be used to assist in puncturing from the inferior vena cava into the adjacent aorta, wherein such puncture may be made either mechanically or electrosurgically.

At block 1110, the process 1100 involves implanting an arterial compliant fluid container 120 into the vena cava vessel 19. Deploying the arterial compliant fluid container 120 may involve anchoring the container in the vessel. Such anchor features or means may be a first port structure of the compliance restoration device 100 being attached against and/or to the wall of the vena cava vessel 19.

In some examples, the anchor(s) is/are configured to be attached to the arterial wall in some manner and/or embedded therein, or may simply serve to present a diameter for the port structure of the implant device that is greater than the opening such as to prevent the device from being pulled back through the opening. Over time, tissue ingrowth may secure the anchor(s) to the arterial wall.

At block 1112, the process 1100 optionally involves attaching the venous compliant fluid container 110 to the arterial compliant fluid container 120 via a conduit 130. In some examples, the compliance restoration device 100 is a single piece and attaching the fluid containers is not needed. In other examples, the compliance restoration device 100 is made up of separate parts that are attached once implanted into the body.

In one scenario, the venous compliant fluid container 110 and the arterial compliant fluid container 120 are implanted and attached in a single operation. In another scenario, the compliant fluid containers are implanted in one operation, with the conduit being attached to the containers during a second operation. By waiting between operations, tissue ingrowth can incur and more securely anchor the compliant fluid containers 110, 120 to the vessel walls.

At block 1114, the process 1100 involves filling the venous compliant fluid container 110 and the arterial compliant fluid container 120 with fluid. In one example, at least one of the compliant fluid containers includes a fill valve for inserting a needle. Fluid is then transmitted through the needle and into the compliance restoration device 100. As the fluid containers are in fluid communication via the conduit 130, filling one of the fluid containers will fill the other one, Other examples may reproduce the utility of the fill valve using various structures, such as using a self-sealing surface that reseals after the needle is withdrawn or patching the needle hole with adhesive material.

Various types of fluid can be used to fill the compliance restoration device 100. One possible fluid is saline. If the compliance restoration device 100 should leak, the body can absorb the saline solution without health risks. Other possibilities include types of silicone fluid with the appropriate viscosity. Some implementations may use a gas, such as CO2 or other inert gas, as the fluid in air-tight fluid containers. By filling the compliance restoration device 100 after implantation, the implantation becomes easier as the unfilled compliance restoration device 100 can be compressed into a much smaller space, allowing smaller openings to be made in the blood vessels.

At block 1116, the process 1100 involves withdrawing the delivery system component(s), such as a catheter/sheath, used to implant the compliance restoration device 100 from the body. In one scenario, the delivery system component(s) are pulled out in the reverse of the entry path used to reach the target implant destination.

### ADDITIONAL DESCRIPTION OF EXAMPLES

Provided below is a list of examples, each of which may include aspects of any of the other examples disclosed herein. Furthermore, aspects of any example described above may be implemented in any of the numbered examples provided below.

Example 1: A compliance restoration implant device comprising: a first compliant fluid container having a first lumen, the first compliant fluid container configured such that the first lumen widens in response to fluid leaving the first compliant fluid container and narrows in response to the fluid entering the first compliant fluid container; a second compliant fluid container having a second lumen, the second compliant fluid container configured such that the second lumen widens in response to the fluid leaving the second compliant fluid container and narrows in response to the fluid entering the second compliant fluid container; and a conduit structure coupled to the first compliant fluid container and the second compliant fluid container, the conduit structure configured to: pass fluid from the first compliant fluid container to the second compliant fluid container in response to a pressure level outside of the second compliant fluid container decreasing; and pass the fluid from the second compliant fluid container to the first compliant fluid container in response to the pressure level outside of the second compliant fluid container increasing.

Example 2: The compliance restoration implant device of any example herein, in particular example 1, further comprising: a first port structure formed on an outer wall of the first compliant fluid container and configured to provide fluid access to an interior of the first compliant fluid container; and a second port structure formed on an outer wall of the first compliant fluid container and configured to provide fluid access to an interior of the second compliant fluid container; wherein the conduit structure is configured to connect to the first port structure and the second port structure, the conduit structure forming a fluid channel between the interior of the first compliant fluid container and the second compliant fluid container.

Example 3: The compliance restoration implant device of any example herein, in particular example 2, wherein the first port structure comprises a stent frame.

Example 4: The compliance restoration implant device of example 1, wherein the first compliant fluid container includes: a first outer wall; a first inner wall; and a first inner fluid reservoir between the first outer wall and the first inner wall; wherein the first inner wall circumferentially forms the first lumen, the first lumen formed longitudinally around the center of the first compliant fluid container.

Example 5: The compliance restoration implant device of any example herein, in particular example 4, wherein the second compliant fluid container includes: a second outer wall; a second inner wall; and a second inner fluid reservoir between the second outer wall and the second inner wall; wherein the second inner wall circumferentially forms the second lumen, the second lumen formed longitudinally around the center of the second compliant fluid container.

Example 6: The compliance restoration implant device of any example herein, in particular example 5, wherein the first compliant fluid container is configured to be placed in a vena cava vessel comprising of one of an inferior vena cava and a superior vena cava.

Example 7: The compliance restoration implant device of any example herein, in particular example 6, wherein the second compliant fluid container is configured to be placed in an aorta.

Example 8: The compliance restoration implant device of any example herein, in particular example 7, wherein the first outer wall of the first compliant fluid container abuts against an inner surface of the vena cava vessel and the first lumen forms a first pathway through the first compliant fluid container for blood flowing through the vena cava vessel.

Example 9: The compliance restoration implant device of any example herein, in particular example 8, wherein the second outer wall of the second compliant fluid container abuts against an inner surface of the aorta and the second lumen forms a second pathway through the second compliant fluid container for blood flowing through the aorta.

Example 10: The compliance restoration implant device of any example herein, in particular examples 7-9, wherein high pressure in the aorta causes the fluid to transfer from the second compliant fluid container to the first compliant fluid container; and low pressure in the aorta causes the fluid to transfer from the first compliant fluid container to the second compliant fluid container.

Example 11: The compliance restoration implant device of any example herein, in particular examples 1-10, wherein a first diameter of the first lumen is configured to increase in response to a decrease in a second diameter of the second lumen; and the first diameter of the first lumen is configured to decrease in response to an increase in the second diameter of the second lumen.

Example 12: The compliance restoration implant device of any example herein, in particular examples 1-11, wherein the fluid comprises a saline solution.

Example 13: The compliance restoration implant device of any example herein, in particular examples 1-12, wherein the first compliant fluid container and the second compliant fluid container are double-walled, tubular sleeves.

Example 14: A compliance restoration implant device comprising: a first compliant fluid container having a first lumen, the first compliant fluid container configured such that the first lumen widens in response to fluid leaving the first compliant fluid container and narrows in response to the fluid entering the first compliant fluid container; a second compliant fluid container having a second lumen, the second compliant fluid container configured such that the second lumen widens in response to the fluid leaving the second compliant fluid container and narrows in response to the fluid entering the second compliant fluid container; and a first port structure on an outer wall of the first compliant fluid container and configured to provide fluid access to an interior of the first compliant fluid container; and a second port structure on an outer wall of the second compliant fluid container and configured to provide fluid access to an interior of the second compliant fluid container.

Example 15: The compliance restoration implant device of any example herein, in particular example 14, further comprising: a conduit structure configured to be coupled to the first port structure of the first compliant fluid container and the second port structure of the second compliant fluid container, the conduit structure configured to form a fluid channel between the first compliant fluid container and the second compliant fluid container by: passing fluid from the first compliant fluid container to the second compliant fluid container in response to a pressure level outside of the second compliant fluid container decreasing; and passing the fluid from the second compliant fluid container to the first compliant fluid container in response to the pressure level outside of the second compliant fluid container increasing.

Example 16: The compliance restoration implant device of any example herein, in particular example 14 or example 15, wherein the first compliant fluid container includes: a first outer wall; a first inner wall; and a first inner fluid reservoir between the first outer wall and the first inner wall; wherein the first inner wall circumferentially forms the first lumen, the first lumen formed longitudinally around the center of the first compliant fluid container.

Example 17: The compliance restoration implant device of any example herein, in particular example 16, wherein the second compliant fluid container includes: a second outer wall; a second inner wall; and a second inner fluid reservoir between the second outer wall and the second inner wall; wherein the second inner wall circumferentially forms the second lumen, the second lumen formed longitudinally around the center of the second compliant fluid container.

Example 18: The compliance restoration implant device of any example herein, in particular example 17, wherein the first compliant fluid container is configured to be placed in a vena cava vessel comprising of one of an inferior vena cava and a superior vena cava.

Example 19: The compliance restoration implant device of any example herein, in particular example 18, wherein the second compliant fluid container is configured to be placed in an aorta.

Example 20: The compliance restoration implant device of any example herein, in particular example 19 wherein the first outer wall of the first compliant fluid container abuts against an inner surface of the vena cava vessel and the first lumen forms a first pathway through the first compliant fluid container for blood flowing through the vena cava vessel.

Example 21: The compliance restoration implant device of any example herein, in particular example 20, wherein the second outer wall of the second compliant fluid container abuts against an inner surface of the aorta and the second lumen forms a second pathway through the second compliant fluid container for blood flowing through the aorta.

Example 22: The compliance restoration implant device of any example herein, in particular example 21, wherein: high pressure in the aorta causes the fluid to transfer from the second compliant fluid container to the first compliant fluid container; and high pressure in the vena cava vessel causes the fluid to transfer from the first compliant fluid container to the second compliant fluid container.

Example 23: The compliance restoration implant device of any example herein, in particular examples 19-22, wherein: a first diameter of the first lumen is configured to increase in response to a decrease in a second diameter of the second lumen; and the first diameter of the first lumen is configured to decrease in response to an increase in the second diameter of the second lumen.

Example 24: The compliance restoration implant device of any example herein, in particular examples 14-23, wherein the fluid comprises a saline solution.

Example 25: The compliance restoration implant device of any example herein, in particular examples 14-24, wherein the first port structure comprises a stent frame.

Example 26: The compliance restoration implant device of any example herein, in particular examples 14-25, wherein the first compliant fluid container and the second compliant fluid container are double-walled, tubular sleeves.

Example 27: A method of inserting a compliance restoration implant device into blood vessels, the method comprising: forming a first opening in a wall of a first blood vessel and a second opening in a wall of a second blood vessel; placing a first compliant fluid container within the first blood vessel; anchoring a first port structure of the first compliant fluid container to the wall of the first blood vessel such that the first port structure provides fluid access, via a conduit, to the first compliant fluid container; placing a second compliant fluid container within the second blood vessel; and anchoring a second port structure of the second compliant fluid container to the wall of the second blood vessel such that the second port structure provides fluid access, via the conduit, to the second compliant fluid container.

Example 28: The method of any example herein, in particular example 27, further comprising: attaching the conduit to the first port structure of the first compliant fluid container and the second port structure of the second compliant fluid container.

Example 29: The method of any example herein, in particular example 27, further comprising: inserting fluid into the first compliant fluid container and the second compliant fluid container.

Example 30: A method of restoring compliance for blood vessels using a compliance restoration implant device, the compliance restoration implant device comprising an arterial fluid container deposed within an aorta and a venous fluid container deposed within in a vena cava vessel, the vena cava vessel including one of an inferior vena cava and a superior vena cava, the method comprising: during diastole in the aorta, expanding the arterial fluid container of the compliance restoration implant device by receiving fluid, via a conduit of the compliance restoration implant device; during diastole in the vena cava vessel, compressing the venous fluid container of the compliance restoration implant device by sending the fluid to the arterial fluid container via the conduit; during systole in the aorta, compressing the arterial fluid container of the compliance restoration implant device by sending the fluid, via the conduit, to the venous fluid container; and during systole in the vena cava vessel, expanding the venous fluid container of the compliance restoration implant device by receiving fluid from the arterial fluid container via the conduit.

Example 31: The method of any example herein, in particular example 30, wherein: the arterial fluid container includes a first lumen, the arterial fluid container configured such that the first lumen widens in response to fluid leaving the arterial fluid container and narrows in response to the fluid entering the arterial fluid container; and the venous fluid container includes a second lumen, the venous fluid container configured such that the second lumen narrows in response to the fluid leaving the venous fluid container and widens in response to the fluid entering the venous fluid container.

Example 32: The method of any example herein, in particular example 31, wherein the arterial fluid container includes: a first outer wall; a first inner wall; and a first inner fluid reservoir between the first outer wall and the first inner wall; wherein the first inner wall circumferentially forms the first lumen, the first lumen formed longitudinally around the center of the arterial fluid container.

Example 33: The method of any example herein, in particular example 32, wherein the venous fluid container includes: a second outer wall; a second inner wall; and a second inner fluid reservoir between the second outer wall and the second inner wall; wherein the second inner wall circumferentially forms the second lumen, the second lumen formed longitudinally around the center of the venous fluid container.

Example 34: The method of any example herein, in particular example 33, wherein the first outer wall of the arterial fluid container abuts against an inner surface of the vena cava vessel and the first lumen forms a first pathway through the arterial fluid container for blood flowing through the vena cava vessel.

Example 35: The method of any example herein, in particular example 34, wherein the second outer wall of the venous fluid container abuts against an inner surface of the aorta and the second lumen forms a second pathway through the venous fluid container for blood flowing through the aorta.

Example 36: The method of any example herein, in particular examples 30-35, wherein the arterial fluid container and the venous fluid container are double-walled, tubular sleeves.

Example 37: The method of any example herein, in particular examples 30-36, wherein the arterial fluid container and the venous fluid container comprise an elastomeric polymer.

Example 38: The method of any example herein, in particular examples 30-37, wherein pressure external to the arterial fluid container is greater during systole than during diastole.

Example 39: The method of any example herein, in particular example 38, wherein a pressure differential between the arterial fluid container and the venous fluid container is greater during systole than during diastole.

Example 40: The method of any example herein, in particular example 39, wherein a greater pressure differential during systole causes the fluid to flow from the arterial fluid container to the venous fluid container.

Example 41: The method of any example herein, in particular example 40, wherein a lesser pressure differential during diastole causes the fluid to flow from the venous fluid container to the arterial fluid container.

Example 42: The method of any example herein, in particular example 41, wherein the venous fluid container comprises a material that is configured to compress against the fluid contained within.

Example 43: The method of any example herein, in particular example 42, wherein the lesser pressure differential during diastole and the material that is configured to compress causes the fluid to flow from the venous fluid container to the arterial fluid container.

The above method(s) can be performed on a living animal or on a simulation, such as on a cadaver, cadaver heart, anthropomorphic ghost, simulator (e.g., with body parts, heart, tissue, etc. being simulated.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

It should be appreciated that in the above description of examples, various features are sometimes grouped together in a single example, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

What is claimed is:

1. A compliance restoration implant device comprising:
a first compliant fluid container having a first lumen that widens in response to fluid leaving the first compliant fluid container and narrows in response to the fluid entering the first compliant fluid container;
a second compliant fluid container having a second lumen that widens in response to the fluid leaving the second compliant fluid container and narrows in response to the fluid entering the second compliant fluid container; and
a conduit structure coupled to the first compliant fluid container and the second compliant fluid container, and that passes fluid from the first compliant fluid container to the second compliant fluid container in response to a pressure level outside of the second compliant fluid container decreasing, and that passes the fluid from the second compliant fluid container to the first compliant fluid container in response to the pressure level outside of the second compliant fluid container increasing.

2. The compliance restoration implant device of claim 1, wherein:
the first compliant fluid container includes:
a first outer wall,
a first inner wall, and
a first inner fluid reservoir between the first outer wall and the first inner wall,
wherein the first inner wall circumferentially forms the first lumen, the first lumen formed longitudinally around the center of the first compliant fluid container; and
the second compliant fluid container includes:
a second outer wall,
a second inner wall, and
a second inner fluid reservoir between the second outer wall and the second inner wall,
wherein the second inner wall circumferentially forms the second lumen, the second lumen formed longitudinally around the center of the second compliant fluid container.

3. The compliance restoration implant device of claim 2, wherein:
the first compliant fluid container is sized to be placed in a vena cava vessel comprising of one of an inferior vena cava and a superior vena cava, the first outer wall of the first compliant fluid container abutting against an inner surface of the inferior or superior vena cava vessel and the first lumen forming a first pathway through the first compliant fluid container for blood flowing through the inferior or superior vena cava vessel; and
the second compliant fluid container is sized to be placed in an aorta, the second outer wall of the second compliant fluid container abutting against an inner surface of the aorta and the second lumen forming a second pathway through the second compliant fluid container for blood flowing through the aorta.

4. The compliance restoration implant device of claim 1, wherein:
a first diameter of the first lumen increases in response to a decrease in a second diameter of the second lumen; and
the first diameter of the first lumen decreases in response to an increase in the second diameter of the second lumen.

5. The compliance restoration implant device of claim 1, wherein the first compliant fluid container and the second compliant fluid container each comprises bovine pericardial tissue, and the device further comprises a respective memory metal frame secured around each of the first and second compliant fluid containers.

6. A compliance restoration implant device comprising:
a first compliant fluid container having a first lumen that widens in response to fluid leaving the first compliant fluid container and narrows in response to the fluid entering the first compliant fluid container;
a second compliant fluid container having a second lumen that widens in response to the fluid leaving the second compliant fluid container and narrows in response to the fluid entering the second compliant fluid container;
a first port structure on an outer wall of the first compliant fluid container to provide fluid access to an interior of the first compliant fluid container; and
a second port structure on an outer wall of the second compliant fluid container to provide fluid access to an interior of the second compliant fluid container.

7. The compliance restoration implant device of claim 6, further comprising:
a conduit structure coupled to the first port structure of the first compliant fluid container and the second port structure of the second compliant fluid container, the conduit structure forming a fluid channel between the first compliant fluid container and the second compliant fluid container by:
passing fluid from the first compliant fluid container to the second compliant fluid container in response to a pressure level outside of the second compliant fluid container decreasing; and
passing the fluid from the second compliant fluid container to the first compliant fluid container in response to the pressure level outside of the second compliant fluid container increasing.

8. The compliance restoration implant device of claim 6, wherein:
the first compliant fluid container is sized to be placed in a vena cava vessel and the first compliant fluid container includes:
a first outer wall,
a first inner wall, and
a first inner fluid reservoir between the first outer wall and the first inner wall,
wherein the first inner wall circumferentially forms the first lumen, the first lumen formed longitudinally around the center of the first compliant fluid container; and
the second compliant fluid container is sized to be placed in an aorta and the second compliant fluid container includes:
a second outer wall,
a second inner wall, and
a second inner fluid reservoir between the second outer wall and the second inner wall,
wherein the second inner wall circumferentially forms the second lumen, the second lumen formed longitudinally around the center of the second compliant fluid container.

9. The compliance restoration implant device of claim 6, wherein:
a first diameter of the first lumen increases to a widened state in response to a decrease in a second diameter of the second lumen; and
the first diameter of the first lumen decreases to a narrowed state in response to an increase in the second diameter of the second lumen, the narrowed state not occluding the first lumen.

10. The compliance restoration implant device of claim 6, wherein the first port structure and the second port structure each comprises a respective woven memory metal braided stent structure.

11. The compliance restoration implant device of claim 6, wherein the first port structure defines a first flow channel having a first diameter and the second port structure defines a second flow channel having a second diameter, the first diameter being greater than the second diameter.

12. The compliance restoration implant device of claim 11, wherein the first diameter is about 2 centimeters (cm) to about 3 centimeters (cm), and the second diameter is about 1 centimeters (cm) to about 2 centimeters (cm).

13. A compliance restoration implant device comprising:
a first compliant fluid container sized to be disposed within a vena cava vessel and having a first lumen to allow flow of blood therethrough away from a heart;
a second compliant fluid container in fluid communication with the first compliant fluid container and sized to be disposed within an aorta, the second compliant fluid container having a second lumen to allow flow of blood therethrough to the heart; and
a conduit structure extending between the first and second compliant fluid containers to provide the fluid communication between the first and second compliant fluid containers, the first and second compliant fluid containers alternating between a respective expanded state and a respective contracted state as fluid is passed between the first and second compliant fluid containers through the conduit structure.

14. The compliance restoration implant device of claim 13, wherein the device:
passes fluid from the second compliant fluid container to the first compliant fluid container in response to a pressure external to the second compliant fluid container pressing upon the second compliant fluid container level; and
returns fluid from the first compliant fluid container to the second compliant fluid container in response to a decrease in the pressure external to the second compliant fluid container pressing upon the second compliant fluid container.

15. The compliance restoration implant device of claim 13, wherein:
the first compliant fluid container has a first tubular configuration and comprises a first inner wall at least partially defining the first lumen, the first lumen narrowing as the first compliant fluid container expands; and
the second compliant fluid container has a second tubular configuration and comprises a second inner wall at least partially defining the second lumen, the second lumen narrowing as the second compliant fluid container expands.

16. The compliance restoration implant device of claim 13, wherein the first and second compliant fluid containers and the conduit structure form an integral piece.

17. The compliance restoration implant device of claim 13, wherein the first and second compliant fluid containers and each distal end of the conduit structure form a respective T-shaped intersection.

18. The compliance restoration implant device of claim 13, further comprising a memory metal frame around the second compliant fluid container.

19. The compliance restoration implant device of claim 13, wherein the fluid comprises saline.

20. The compliance restoration implant device of claim 13, wherein the fluid comprises a silicone fluid.
